(19) United States
(12) Patent Application Publication
(10) Pub. No.: US 2022/0137035 A1
(43) Pub. Date: May 5, 2022

(54) DETECTION OF HEMOGLOBIN A1C (HbA1c) IN BLOOD

(71) Applicant: Procisedx Inc., San Diego, CA (US)

(72) Inventors: Larry Mimms, San Diego, CA (US); Mark Renshaw, San Diego, CA (US); Kevin Chon, San Diego, CA (US); Michael Hale, San Diego, CA (US)

(73) Assignee: Procisedx Inc., San Diego, CA (US)

(21) Appl. No.: 17/521,747

(22) Filed: Nov. 8, 2021

Related U.S. Application Data
(63) Continuation of application No. PCT/US2020/032823, filed on May 14, 2020.
(60) Provisional application No. 62/858,243, filed on Jun. 6, 2019.

(51) Int. Cl. G01N 33/542; G01N 33/72

(52) U.S. Cl. CPC G01N 33/542; G01N 33/72

(57) ABSTRACT

An assay method for detecting and measuring the presence or amount of glycated hemoglobin in a sample using fluorescence resonance energy transfer (FRET).

### CROSS-REFERENCES TO RELATED APPLICATIONS

This application is a continuation of PCT/US2020/032823, filed May 14, 2020, which claims priority to U.S. Provisional Application No. 62/858,243, filed Jun. 6, 2019, the disclosures of which are hereby incorporated by reference in their entireties for all purposes.

### BACKGROUND

Glycated hemoglobin (HbA1c) is a hemoglobin-glucose combination formed non-enzymatically within the cell. Over time, the glucose becomes covalently bound to the hemoglobin molecule. This glycan hemoglobin provides a time-average amount of blood glucose concentration through the 120-day life span of the red blood cell. Thus, glycated hemoglobin levels provide an objective measurement of blood glucose control over time.

A number of analytical techniques are used to measure HbA1c. For example, clinical laboratories use high-performance liquid chromatography, immunoassay, enzymatic assays, capillary electrophoresis and affinity chromatography. As the average amount of blood glucose increases, the fraction of glycosolated hemoglobin increases in a predictable way. Therefore, the percentage of HbA1c in blood can serve as a marker for average blood glucose level over the past three months and thus, it can be used to diagnose diabetes.

Glycated hemoglobin testing is recommended for checking blood sugar in people who might be pre-diabetic. In fact, the American Diabetes Association (ADA) added the blood concentration of glycated hemoglobin (HbA1c) of over 6.5% as another criterion for the diagnosis of diabetes. Screening of elevated HbA1c level to a broader population represents an effective way for early diagnosis of diabetes. Higher amounts of HbA1c not only indicate poorer control of blood glucose levels, but is also associated with cardiovascular disease, nephropathy, and retinopathy. This emphasizes the importance of the precise and accurate monitoring of HbA1c. Furthermore, monitoring HbA1c in type-1 diabetic patients may improve treatment.

In view of the foregoing, there is a need in the art for new more precise and accurate ways to measure glycated hemoglobin HbA1c. The present disclosure provides this and other needs.

### BRIEF SUMMARY

The present disclosure relates to methods for detecting glycated human hemoglobin in, for example, human whole blood, that are precise and accurate and allow for monitoring in diabetic patients. Diabetes mellitus is a life-long metabolic disease that can cause several complications representing one of the most important health concerns in today's society. The early diagnosis of diabetes and regular monitoring of blood glucose level are essential factors in preventing the health complications resulting from this disease.

In certain aspects, this disclosure provides methods for the determination of the percentage of glycated hemoglobin in a blood sample. In certain instances, there is a separate measurement for the amount of total hemoglobin in the sample.

As such, in one embodiment, the present disclosure provides a method for measuring the amount of glycated hemoglobin (HbA1c) in a sample, the method comprising:
contacting the sample with an anti-hemoglobin (HbA₀) antibody labeled with a first fluorophore, wherein the anti-hemoglobin (HbA₀) antibody also binds glycated hemoglobin (HbA1c);
contacting the sample with an anti-glycated hemoglobin (HbA1c) antibody labeled with a second fluorophore;
incubating the sample for a time sufficient to obtain a dual labeled glycated hemoglobin (HbA1c); and
exciting the sample have dual labeled glycated hemoglobin (HbA1c) using a light source to detect a fluorescence emission signal associated with fluorescence resonance energy transfer (FRET).

In certain aspects, the glycated hemoglobin (HbA1c) amount is a percent of total hemoglobin. The amount of total hemoglobin can be calculated using a variety of methods.

In another embodiment, the present disclosure provides a method for measuring the amount of glycated hemoglobin (HbA1c) in vitro in a sample, the method comprising:
obtaining a sample from a subject;
contacting the sample with an anti-hemoglobin (HbA₀) antibody labeled with a first fluorophore, wherein the anti-hemoglobin (HbA₀) antibody also binds glycated hemoglobin (HbA1c);
contacting the sample with an anti-glycated hemoglobin (HbA1c) antibody labeled with a second fluorophore;
incubating the sample for a time sufficient to obtain a dual labeled glycated hemoglobin (HbA1c); and
exciting the sample have dual labeled glycated hemoglobin (HbA1c) using a light source to detect fluorescence emission signal associated with fluorescence resonance energy transfer (FRET), to determine the amount of glycated hemoglobin (HbA1c) in the sample.

In certain aspects, the first fluorophore is a FRET donor.

In certain aspects, the second fluorophore is a FRET acceptor.

These and other aspects, objects and embodiments will become more apparent when read with the detailed description and figures that follow.

### BRIEF DESCRIPTION OF THE DRAWINGS

FIGS. 1A-B illustrate one embodiment of the present disclosure showing an assay format of a HbA1c assay. As shown in FIG. 1A, the HbA₀ antibody labeled with a donor fluorophore can bind to both HbA₀ and HbA1c. When a HbA1c specific antibody, which is labeled with an acceptor fluorophore, binds simultaneously with the HbA₀ antibody to HbA1c, a FRET signal occurs; individual reagents are shown in FIG. 1B.

FIGS. 2A-B illustrate standard curves generated using methods of the present disclosure. The x axis is an ERM standard % A1c and they axis is the calculated HbA1c % using methods of the present disclosure.

FIG. 3 illustrates one embodiment of a donor of the present disclosure.

FIG. 4 illustrates donor and acceptor wavelengths in one embodiment of the present disclosure. Tb-H22TRENIAM-5LIO-NHS emission profile is shown (490 nm, 545 nm, 580 nm and 620 nm). Acceptor emission peaks are shown in (AF488, second arrow from left), (AF546, fourth arrow from left) and (AF647, seventh arrow from the left i.e., first arrow on the right).

FIG. 5 illustrates one embodiment of an acceptor of the present disclosure.

FIG. 6 illustrates one embodiment of a standard curve of Hct (%) samples determined using fluorescence of a donor fluorophore.

FIG. 7 illustrates one embodiment of a standard curve for hematocrit levels.

FIG. 8 illustrates a correlation of the present methods with an Afinion point of care device.

FIG. 9 illustrates a correlation of the present methods with an Afinion point of care device measured patient sample; BioRad D-100 patient samples; Point Scientific standards and Lyphocheck standards.

### DETAILED DESCRIPTION

### I. Definitions

The terms “a,” “an,” or “the” as used herein not only includes aspects with one member, but also includes aspects with more than one member.

The term “about” as used herein to modify a numerical value indicates a defined range around that value. If “X” were the value, “about X” would indicate a value from 0.9× to 1.1×, and more preferably, a value from 0.95× to 1.05×. Any reference to “about X” specifically indicates at least the values X, 0.95×, 0.96×, 0.97×, 0.98×, 0.99×, 1.01×, 1.02×, 1.03×, 1.04×, and 1.05×. Thus, “about X” is intended to teach and provide written description support for a claim limitation of, e.g., “0.98×.”

When the modifier “about” is applied to describe the beginning of a numerical range, it applies to both ends of the range. Thus, “from about 500 to 850 nm” is equivalent to “from about 500 nm to about 850 nm.” When “about” is applied to describe the first value of a set of values, it applies to all values in that set. Thus, “about 580, 700, or 850 nm” is equivalent to “about 580 nm, about 700 nm, or about 850 nm.”

“Activated acyl” as used herein includes a —C(O)-LG group. “Leaving group” or “LG” is a group that is susceptible to displacement by a nucleophilic acyl substitution (i.e., a nucleophilic addition to the carbonyl of —C(O)-LG, followed by elimination of the leaving group). Representative leaving groups include halo, cyano, azido, carboxylic acid derivatives such as t-butylcarboxy, and carbonate derivatives such as i-BuOC(O)O—. An activated acyl group may also be an activated ester as defined herein or a carboxylic acid activated by a carbodiimide to form an anhydride (preferentially cyclic) or mixed anhydride —OC(O)R^{a} or —OC(NR^{a})NHR^{b} (preferably cyclic), wherein R^{a} and R^{b} are members independently selected from the group consisting of C₁-C₆ alkyl, C₁-C₆ perfluoroalkyl, C₁-C₆ alkoxy, cyclohexyl, 3-dimethylaminopropyl, or N-morpholinoethyl. Preferred activated acyl groups include activated esters.

“Activated ester” as used herein includes a derivative of a carboxyl group that is more susceptible to displacement by nucleophilic addition and elimination than an ethyl ester group (e.g., an NHS ester, a sulfo-NHS ester, a PAM ester, or a halophenyl ester). Representative carbonyl substituents of activated esters include succinimidyloxy (—OC₄H₄NO₂), sulfosuccinimidyloxy (—OC₄H₃NO₂SO₃H), -1-oxybenzotriazolyl (—OC₆H₄N₃); 4-sulfo-2,3,5,6-tetrafluorophenyl; or an aryloxy group that is optionally substituted one or more times by electron-withdrawing substituents such as nitro, fluoro, chloro, cyano, trifluoromethyl, or combinations thereof (e.g., pentafluorophenyloxy, or 2,3,5,6-tetrafluorophenyloxy). Preferred activated esters include succinimidyloxy, sulfosuccinimidyloxy, and 2,3,5,6-tetrafluorophenyloxy esters.

“FRET partners” refer to a pair of fluorophores consisting of a donor fluorescent compound such as cryptate and an acceptor compound such as Alexa 647, when they are in proximity to one another and when they are excited at the excitation wavelength of the donor fluorescent compound, these compounds emit a FRET signal. It is known that, in order for two fluorescent compounds to be FRET partners, the emission spectrum of the donor fluorescent compound must partially overlap the excitation spectrum of the acceptor compound. The preferred FRET-partner pairs are those for which the value R0 (Forster distance, distance at which energy transfer is 50% efficient) is greater than or equal to 30 Å.

“Fluorescence resonance energy transfer (FRET)” or “Førster resonance energy transfer (FRET)” refer to a mechanism describing energy transfer between a donor compound such as cryptate and an acceptor compound such as Alexa 647, when they are in proximity to one another and when they are excited at the excitation wavelength of the donor fluorescent compound. A donor compound, initially in its electronic excited state, may transfer energy to an acceptor fluorophore through nonradiative dipole-dipole coupling. The efficiency of this energy transfer is inversely proportional to the sixth power of the distance between donor and acceptor, making FRET extremely sensitive to small changes in distance.

“FRET signal” refers to any measurable signal representative of FRET between a donor fluorescent compound and an acceptor compound. A FRET signal can therefore be a variation in the intensity or in the lifetime of luminescence of the donor fluorescent compound or of the acceptor compound when the latter is fluorescent.

“Hemoglobin” refers to a hemeprotein consisting of two of each of the two types of subunits, the α-chain and the β-chain, and has a molecular weight of 64,000. The sequence of the three amino acids at the N terminus of the α-chain of hemoglobin is valine-leucine-serine and the sequence of the three amino acids at the N terminus of the β-chain is valine-histidine-leucine. Hemoglobin is the iron-containing oxygen transport metalloprotein in the red blood cells. Hemoglobin's structure consists of a tetramer of two pairs of protein molecules: two α globin chains and two non-α globin chains. The α globin genes are HbA1 and HbA2. The normal adult hemoglobin molecule (HbA) consists of two α and two β chains (α₂β₂), and makes up about 97% of most normal human adult hemoglobin. Other minor hemoglobin components may be formed by posttranslational modification of HbA. These include hemoglobins A1a, A1b, and A1c. Of these, A1c is the most abundant minor hemoglobin component. A1c is formed by the chemical condensation of hemoglobin and glucose which are both present in high concentrations in erythrocytes. This process occurs slowly and continuously over the life span of erythrocytes, which is 120 days on average. Furthermore, the rate of A1c formation is directly proportional to the average concentration of glucose within the erythrocyte during its lifespan. Hence, as levels of chronic hyperglycemia increase, so does the formation of A1c.

As used herein, the term “glycated hemoglobin” or “glycosylated hemoglobin” refer to any form of human hemoglobin to which a glucose molecule has been bound to the amino terminus of the β-chain of the hemoglobin without the action of an enzyme. HbA1c forms through a non-enzymatic reaction in which glucose attaches to the valine amino terminal of one or both chains of hemoglobin A. HbA1c is defined as hemoglobin in which the N-terminal valine residue of the β-chain is particularly glycated; however, hemoglobin is known to have multiple glycation sites within the molecule, including the N terminus of the α-chain (see, The Journal of Biological Chemistry (1980), 256, 3120-3127).

### II. Embodiments

In the normal 120-day lifespan of the red blood cell, glucose molecules react with hemoglobin, which accumulates an adduct known as glycated hemoglobin (HbA1c). As the average amount of blood glucose increases, the fraction of glycosolated or glycalated hemoglobin increases in a predictable way. Thus, the percentage of HbA1c % in blood can serve as a marker for average blood glucose level over the past three months and therefore, it can be used to diagnose diabetes or abnormally high or low blood glucose.

In one embodiment, the present disclosure provides a method for measuring the amount of glycated hemoglobin (HbA1c) in a sample, the method comprising:
contacting the sample with an anti-hemoglobin (HbA₀) antibody labeled with a first fluorophore, wherein the anti-hemoglobin (HbA₀) antibody also binds glycated hemoglobin (HbA1c);
contacting the sample with an anti-glycated hemoglobin (HbA1c) antibody labeled with a second fluorophore;
incubating the sample for a time sufficient to obtain a dual labeled glycated hemoglobin (HbA1c); and
exciting the sample have dual labeled glycated hemoglobin (HbA1c) using a light source to detect fluorescence emission signal associated with fluorescence resonance energy transfer (FRET).

In another embodiment, the present disclosure provides a method for measuring the amount of glycated hemoglobin (HbA1c) in vitro in a sample, the method comprising:
obtaining a sample from a subject;
contacting the sample with an anti-hemoglobin (HbA₀) antibody labeled with a first fluorophore, wherein the anti-hemoglobin (HbA₀) antibody also binds glycated hemoglobin (HbA1c);
contacting the sample with an anti-glycated hemoglobin (HbA1c) antibody labeled with a second fluorophore;
incubating the sample for a time sufficient to obtain a dual labeled glycated hemoglobin (HbA1c); and
exciting the sample have dual labeled glycated hemoglobin (HbA1c) using a light source to detect fluorescence emission signal associated with fluorescence resonance energy transfer (FRET), to determine the amount of glycated hemoglobin (HbA1c) in the sample.

In one aspect, the anti-glycated hemoglobin (HbA1c) antibody labeled with a second fluorophore does not cross-react with hemoglobin (HbA₀), i.e., it is specific to glycated hemoglobin (HbA1c).

In certain aspects, the FRET assay is a time-resolved FRET assay. The fluorescence emission signal or measured FRET signal is directly correlated with the biological phenomenon studied. In fact, the level of energy transfer between the donor compound and the acceptor fluorescent compound is proportional to the reciprocal of the distance between these compounds to the 6th power. For the donor/acceptor pairs commonly used by those skilled in the art, the distance Ro (corresponding to a transfer efficiency of 50%) is in the order of 1, 5, 10, 20 or 30 nanometers.

In certain aspects, the sample is a biological sample. Suitable biological samples include, but are not limited to, whole blood, plasma, serum, blood cells, cell samples, urine, spinal fluid, sweat, tear fluid, saliva, skin, mucous membrane, and hair. As a sample, whole blood, plasma, serum, blood cells and such are preferred, and whole blood, blood cells, and such are particularly preferred. Whole blood includes samples of whole blood-derived blood cell fractions admixed with plasma. With regard to these samples, samples subjected to pretreatments such as hemolysis, separation, dilution, concentration, and purification can be used. In a one aspect, the biological sample is a whole blood or a serum sample.

In certain aspects, the FRET energy donor compound is a cryptate, such as a lanthanide cryptate.

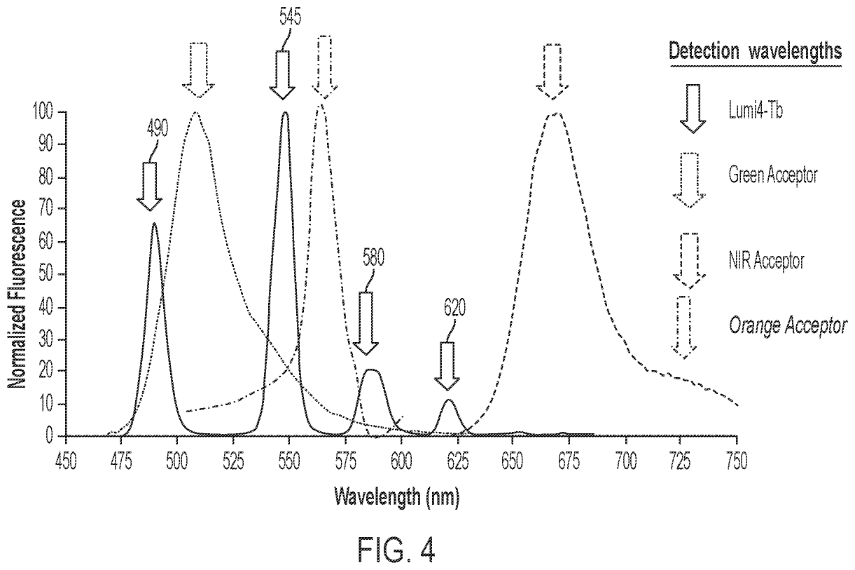

In certain aspects, the cryptate has an absorption wavelength between about 300 nm to about 400 nm such as about 325 nm to about 375 nm. In certain aspects, as shown in FIG. 4, cyptate dyes have four fluorescence emission peaks at about 490 nm, about 548 nm, about 587 nm, and 620 nm. Thus, as a donor, the cryptate is compatible with fluorescein-like (green zone) molecules, Cy5, DY-647-like (red zone) acceptors, Allophycocyanin (APC), or Phycoeruythrin (PE) to perform TR-FRET experiments. Other acceptors include Alexa Fluor 488, Alexa Fluor 546, and Alexa Fluor 647.

In certain aspects of the embodiments, the assay uses a donor fluorophore consisting of terbium bound within a cryptate. The terbium cryptate can be excited with a 365 nm UV LED. The terbium cryptate emits at four (4) wavelengths within the visible region. In one aspect, the assay uses the lowest donor emission energy peak of 620 nm as the donor signal within the assay. In certain aspects, the acceptor fluorophore, when in very close proximity, is excited by the highest energy terbium cryptate emission peak of 490 nm causing light emission at 520 nm. Both the 620 nm and 520 nm emission wavelengths are measured independently in a device or instrument and results can be reported as RFU ratio 620/520.

In certain aspects, the introduction of a time delay between a flash excitation and the measurement of the fluorescence at the acceptor emission wavelength allows to discriminate long lived from short-lived fluorescence and to increase signal-to-noise ratio.

In certain aspects, the methods herein can be used to detect and or diagnose diabetes or prediabetes. Pre-diabetes, also referred to as borderline diabetes, is usually a precursor to diabetes. It occurs when the blood glucose levels are higher than normal, but not high enough for the patient to be considered to have diabetes.

In a one aspect, the biological sample is a whole blood. The blood sample can be an untreated sample. Alternatively, the blood sample may be diluted or processed by concentration or filtration. The blood sample can be a whole blood sample collected using conventional phlebotomy methods.

In a one aspect, the sample includes red blood cells. In certain aspects, the red blood cells are from whole blood. In certain aspects, the red blood cells are lysed. In other aspects, the sample does not include red blood cells.

In a one aspect, the blood sample is treated to lyse the red blood cells. This can be done by diluting a blood sample in a lysing agent, such as deionized distilled water, at a concentration of 1/1 (i.e. 1 part blood to 1 part lysing agent or distilled deionized water). Alternatively, the sample can be frozen to lyse the cells.

In a one aspect, the blood sample is diluted after lysis. The blood sample may be diluted 1/10 (i.e. one part sample in 10 parts diluent), 1/500, 1/1000, 1/200, 1/2500, 1/8000 or more. In one aspect, the sample is diluted 1/2000 i.e. one part blood sample in 2000 parts diluent. In one aspect, the diluent can be water, 0.1% trifluoroacetic acid in distilled deionized water, or distilled deionized water. In one aspect, the blood sample is not processed between lysis and dilution.

In certain instances, the HbA1c levels can be from about 1% to about 12%. Typically for a normal individual, the HbA1c levels are less than about 5.6% such as about 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, or about 5.6%.

In a one aspect, levels of HbA1c just below 6.5% such as 5.7% 5.8, 5.9, 6, 6.1, 6.2, 6.3, 6.4, may indicate the presence of intermediate hyperglycemia.

In certain aspects, HbA1c levels can be used to diagnosis diabetes. Such diagnosis can be made if the HbA1c level is ≥6.5%, such as 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 10.1, 10.2, 10.3, 10.4, 10.5, 10.6, 10.7, 10.8, 10.9, 11, 11.1, 11.2, 11.3, 11.4, 11.5, 11.6, 11.7, 11.8, 11.9, and/or 12% (International Expert Committee report on the role of the A1C assay in the diagnosis of diabetes. Diabetes Care. 2009; 32:1327-1334). In certain instances, diagnosis can be confirmed with a repeat HbA1c test, unless clinical symptoms and plasma glucose levels >11.1 mmol/1(200 mg/dl) are present in which case further testing is not required. Levels of HbA1c just below 6.5% (e.g., 5.7-6.4%) may indicate the presence of intermediate hyperglycaemia. Individuals with a HbA1c level between 6.0 and 6.5% are at particularly high risk and might be considered for diabetes prevention interventions. In contrast to the common plasma glucose tests, the level of glycated hemoglobin is not influenced by daily fluctuations in the blood glucose concentration, but reflect the average glucose levels over the prior six to eight weeks.

The % HbA1c level is a percent of total hemoglobin. Total hemoglobin can be calculated using a variety of methods. For example, total hemoglobin can be calculated using a FRET technique. A first pan anti-hemoglobin antibody labeled with a donor and a second pan anti-hemoglobin antibody labeled with an acceptor will allow for the total amount of hemoglobin present in a sandwich assay. In other instances, total hemoglobin can be measured using an absorption method, CO-oximetry, or estimates from hematocrit levels.

In one aspect, total hemoglobin is measured using an absorption method. The reference method for measuring hemoglobin by the International Committee for Standardization in Hematology (ICSH) (Recommendations for haemoglobinometry in human blood. Br J Haematol. 1967; 13 (suppl:71-6)), is the hemiglobincyanide (HiCN) test, which remains the recommended method of the ICSH. The HiCN test is the test against which all new ctHb methods are judged and standardized. In this method, a blood sample is diluted in a solution containing potassium ferricyanide and potassium cyanide. Potassium ferricyanide oxidizes the iron in heme to the ferric state to form methemoglobin, which is converted to hemiglobincyanide (HiCN) by potassium cyanide. HiCN is a stable colored product, which in solution has an absorbance maximum at 540 nm and obeys Beer-Lambert's law. Absorbance of the diluted sample at 540 nm is compared with absorbance at the same wavelength of a standard HiCN solution whose equivalent hemoglobin concentration is known.

In another aspect, total hemoglobin is measured using a CO-oximetery method. The measurement of ctHb by CO-oximetry is based on the fact that hemoglobin and all its derivatives are colored proteins which absorb light at specific wavelengths and thus have a characteristic absorbance spectrum. Beer-Lambert's law dictates that absorbance of a single compound is proportional to the concentration of that compound. If the spectral characteristic of each absorbing substance in a solution is known, absorbance readings of the solution at multiple wavelengths can be used to calculate the concentration of each absorbing substance. In the CO-oximeter absorbance measurements of a hemolyzed blood sample, light is irradiated at multiple wavelengths across a range that hemoglobin species absorb light (520-620 nm) and software is used to calculate the concentration of each of the hemoglobin derivatives (HHb, O₂Hb, MetHb and COHb). Total hemoglobin (ctHb) is the calculated sum of these derivatives.

In yet another aspect, it is possible to calculate the total Hb concentration by measuring the amount of hematocrit (Hct). Hematocrit is the ratio of the volume of packed red blood cells to the total blood volume. It is also known as the packed cell volume, or PCV. In normal conditions there is a linear relationship between hematocrit and the concentration of hemoglobin (ctHb). The relationship can be expressed as follows:

Hct (%)=(0.0485×ctHb (mmol/L)+0.0083)×100

(Kokholm G. Simultaneous measurements of blood pH, pCO2, pO2 and concentrations of hemoglobin and its derivatives—a multicenter study. Radiometer publication AS107. Copenhagen: Radiometer Medical A/S, 1991).

In another embodiment, the present disclosure provides a competitive assay method for detecting and measuring the amount of glycated hemoglobin HbA1c in a sample, the method comprising:
contacting the sample with a complex comprising an anti-hemoglobin (HbA₀) antibody labeled with a first fluorophore, wherein the anti-hemoglobin (HbA₀) antibody also binds glycated hemoglobin (HbA1c), an anti-glycated hemoglobin (HbA1c) antibody labeled with a second fluorophore and an isolated glycated hemoglobin HbA1c, wherein the first or second fluorophore is a FRET donor;
incubating the sample with the complex for a time sufficient for glycated hemoglobin HbA1c in the sample to compete for binding to the anti-glycated hemoglobin (HbA1c) antibody; and
exciting the sample having the complex using a light source to detect a fluorescence emission signal associated with FRET,
wherein an absence of the fluorescence emission signal or a decrease in the fluorescence emission signal relative to the fluorescence emission signal initially emitted by the complex indicates the amount of glycated hemoglobin HbA1c in a sample.

In certain aspects, the first fluorophore is a FRET donor.

In certain aspects, the second fluorophore is a FRET acceptor.

In certain aspects, the methods herein can be used to diagnose diabetes as well as monitor glycemic control in patients with diabetes. The associated detection methods are simple, sensitive, specific, rapid, and cost-effective. A human blood sample when processed using the methods give accurate and rapid results.

1. Cryptates as FRET Donors

In certain aspects, the terbium cryptate molecule “Lumi4-Tb” from Lumiphore, marketed by Cisbio bioassays is used as the cryptate donor. The terbium cryptate “Lumi4-Tb” having the formula below, which can be coupled to an antibody by a reactive group, in this case, for example, an NHS ester:

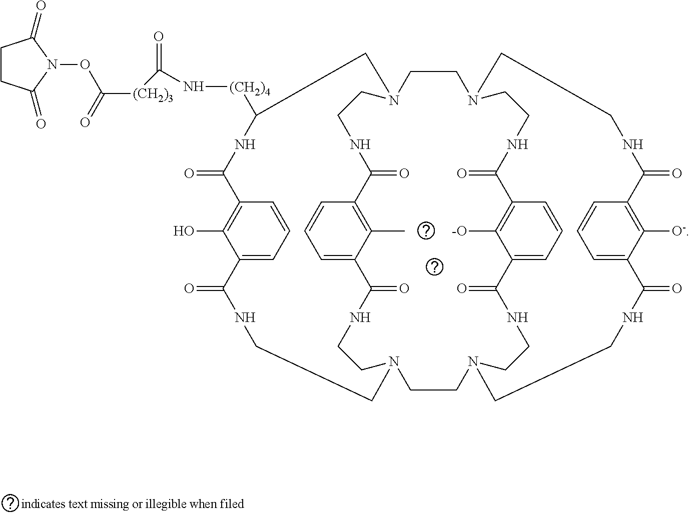

An activated ester (an NHS ester) can react with a primary amine on an antibody to make a stable amide bond. A maleimide on the cryptate and a thiol on the antibody can react together and make a thioether. Alkyl halides react with amines and thiols to make alkylamines and thioethers, respectively. Any derivative providing a reactive moiety that can be conjugated to an antibody can be utilized herein.

In certain aspects, the antibodies used are linked to a fluorophore. Two different fluorophore may be used in the methods of the invention which may be linked to two antibodies binding to i) anti-hemoglobin (HbA₀) antibody, wherein the anti-hemoglobin (HbA0) antibody also binds glycated hemoglobin (HbA1c); and (ii) an anti-glycated hemoglobin (HbA1c) antibody. One fluorophore has longer fluorescence time (donor) than the other fluorophore used (acceptor). The donor can be Lumi4-Tb (Tb²⁺ cryptate) or an Europium cryptate (Eu³⁺ cryptate). The proximity between the donor and acceptor is assessed by detecting the level of energy transfer by measuring the fluorescence emission.

In certain other aspects, cryptates disclosed in WO2015157057, titled “Macrocycles” are suitable for use in the present disclosure. This publication contains cryptate molecules useful for labeling biomolecules. As disclosed therein, certain of the cryptates have the structure as follows:

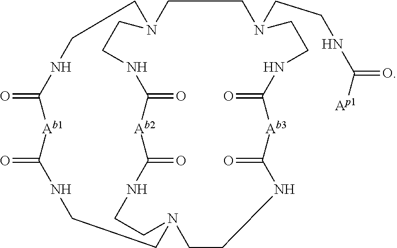

In certain other aspects, a terbium cryptate useful in the present disclosure is shown below:

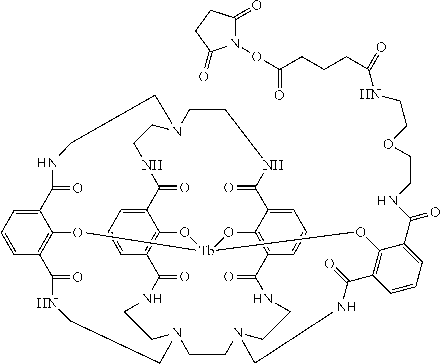

In certain aspects, the cryptates that are useful in the present invention are disclosed in WO 2018/130988, published Jul. 19, 2018. As disclosed therein, the compounds of Formula I are useful as FRET donors in the present disclosure:

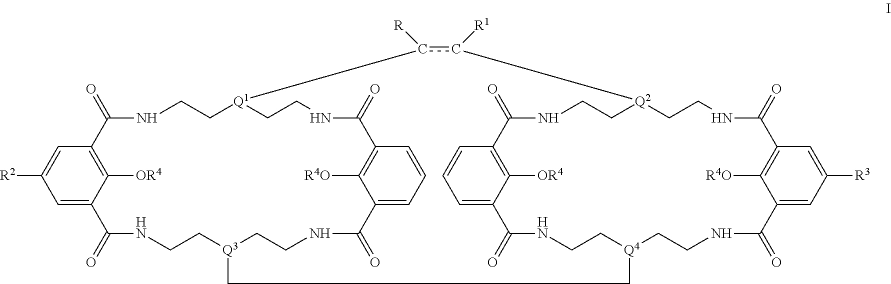

wherein when the dotted line is present, R and R¹ are each independently selected from the group consisting of hydrogen, halogen, hydroxyl, alkyl optionally substituted with one or more halogen atoms, carboxyl, alkoxycarbonyl, amido, sulfonato, alkoxycarbonylalkyl or alkylcarbonylalkoxy or alternatively, R and R¹ join to form an optionally substituted cyclopropyl group wherein the dotted bond is absent;

R² and R³ are each independently a member selected from the group consisting of hydrogen, halogen, SO₃H, —SO₂—X, wherein X is a halogen, optionally substituted alkyl, optionally substituted aryl, optionally substituted alkenyl, optionally substituted alkynyl, optionally substituted cycloalkyl, or an activated group that can be linked to a biomolecule, wherein the activated group is a member selected from the group consisting of a halogen, an activated ester, an activated acyl, optionally substituted alkylsulfonate ester, optionally substituted arylsulfonate ester, amino, formyl, glycidyl, halo, haloacetamidyl, haloalkyl, hydrazinyl, imido ester, isocyanato, isothiocyanato, maleimidyl, mercapto, alkynyl, hydroxyl, alkoxy, amino, cyano, carboxyl, alkoxycarbonyl, amido, sulfonato, alkoxycarbonylalkyl, cyclic anhydride, alkoxyalkyl, a water solubilizing group or L;

R⁴ are each independently a hydrogen, C₁-C₆ alkyl, or alternatively, 3 of the R⁴ groups are absent and the resulting oxides are chelated to a lanthanide cation; and

Q¹-Q⁴ are each independently a member selected from the group of carbon or nitrogen.

2. FRET Acceptors

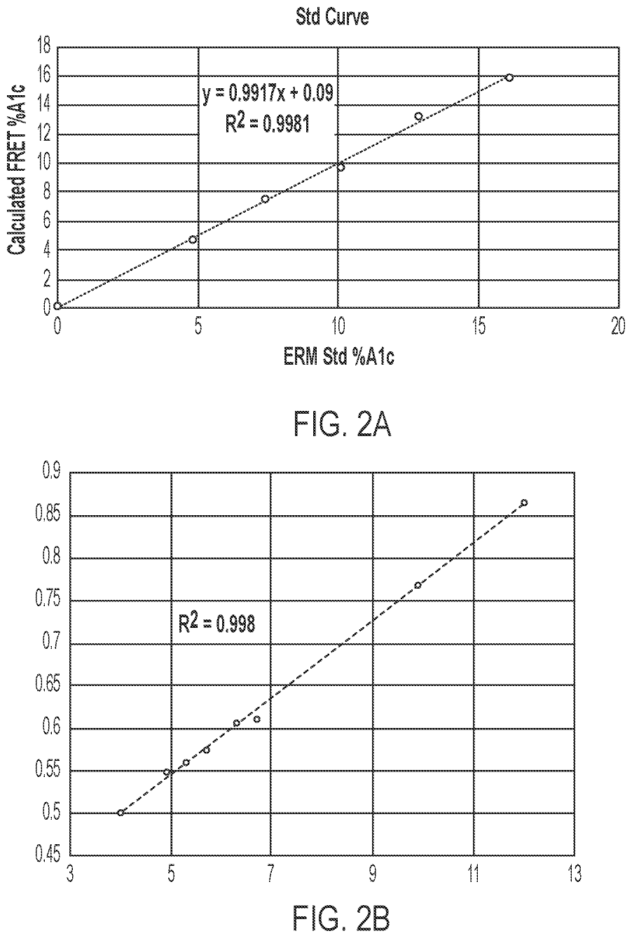

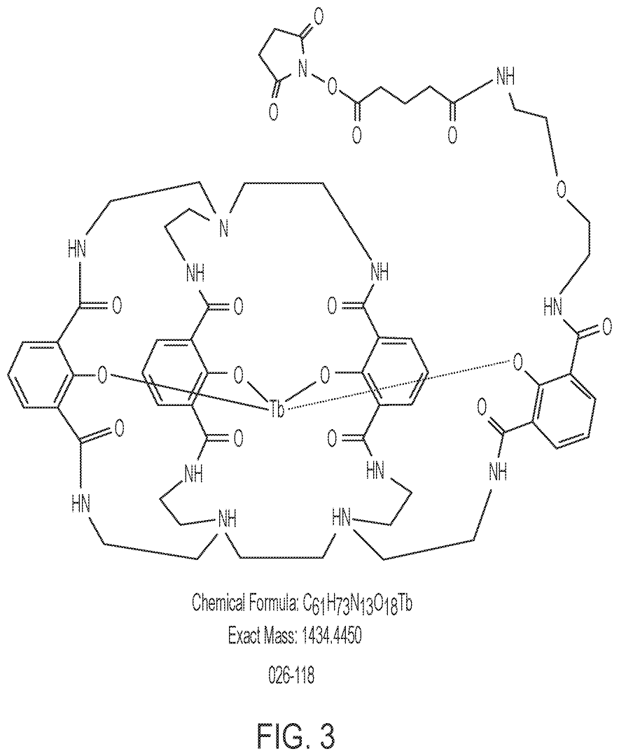

In order to detect a FRET signal, a FRET acceptor is required. The FRET acceptor has an excitation wavelength that overlaps with an emission wavelength of the FRET donor. The FRET signal of the acceptor is proportional to the concentration level of glycated hemoglobin present in the sample, such as a patient's blood sample as interpolated from a known amount of calibrators i.e., a standard curve (FIG. 2). A cryptate donor can be used to label the first antibody AB-1 (FIG. 3). Lumi4 has 4 spectrally distinct peaks, at about 490 nm, about 545 nm, about 580 nm, and about 620 nm, which can be used for energy transfer (FIG. 4). An acceptor can be used to label the second antibody AB-2.

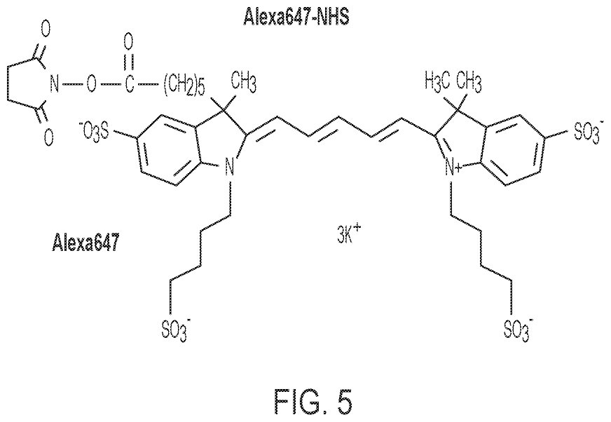

The acceptor molecules that can be used include, but are not limited to, fluorescein-like (green zone) acceptor, Cy5, DY-647, Alexa Fluor 488, Alexa Fluor 546, Allophycocyanin (APC), Phycoeruythrin (PE) and Alexa Fluor 647 (FIG. 5). Donor and acceptor fluorophores having reactive moieties such as an NHS ester can be conjugated using a primary amine on an antibody.

Other acceptors include, but are not limited to, cyanin derivatives, D2, CYS, fluorescein, coumarin, rhodamine, carbopyronine, oxazine and its analogs, Alexa Fluor fluorophores, Crystal violet, perylene bisimide fluorophores, squaraine fluorophores, boron dipyrromethene derivatives, NBD (nitrobenzoxadiazole) and its derivatives, DABCYL (4-((4-(dimethylamino)phenyl)azo)benzoic acid). Further acceptors include XL665, or fluorescein or d2.

In one aspect, fluorescence can be characterized by wavelength, intensity, lifetime, polarization or a combination thereof

3. Antibodies

In certain aspects, an activated ester (an NHS ester) of the donor or acceptor can react with a primary amine on an antibody to make a stable amide bond. For example, a maleimide on the cryptate or the acceptor (e.g., Alexa 647) and a thiol on the antibody can react together and make a thioether. Alkyl halides react with amines and thiols to make alkylamines and thioethers, respectively. Any derivative providing a reactive moiety that can be conjugated to an antibody can be utilized herein to make the first antibody labeled with a donor fluorophore specific for the analyte, as well as, the second antibody labeled with an acceptor fluorophore specific for analyte.

The methods herein can use a variety of samples, which include a tissue sample, blood, biopsy, serum, plasma, saliva, urine, or stool sample.

4. Production of Antibodies

The generation and selection of antibodies not already commercially available can be accomplished several ways. For example, one way is to express and/or purify a polypeptide of interest (i.e., antigen) using protein expression and purification methods known in the art, while another way is to synthesize the polypeptide of interest using solid phase peptide synthesis methods known in the art. See, e.g., Guide to Protein Purification, Murray P. Deutcher, ed., Meth. Enzymol., Vol. 182 (1990); Solid Phase Peptide Synthesis, Greg B. Fields, ed., Meth. Enzymol., Vol. 289 (1997); Kiso et al., Chem. Pharm. Bull., 38:1192-99 (1990); Mostafavi et al., Biomed. Pept. Proteins Nucleic Acids, 1:255-60, (1995); and Fujiwara et al., Chem. Pharm. Bull., 44:1326-31 (1996). The purified or synthesized polypeptide can then be injected, for example, into mice or rabbits, to generate polyclonal or monoclonal antibodies. One skilled in the art will recognize that many procedures are available for the production of antibodies, for example, as described in Antibodies, A Laboratory Manual, Harlow and Lane, Eds., Cold Spring Harbor Laboratory, Cold Spring Harbor, N.Y. (1988). One skilled in the art will also appreciate that binding fragments or Fab fragments which mimic antibodies can also be prepared from genetic information by various procedures (see, e.g., Antibody Engineering: A Practical Approach, Borrebaeck, Ed., Oxford University Press, Oxford (1995); and Huse et al., J. Immunol., 149:3914-3920 (1992)).

In addition, numerous publications have reported the use of phage display technology to produce and screen libraries of polypeptides for binding to a selected target antigen (see, e.g, Cwirla et al., Proc. Natl. Acad. Sci. USA, 87:6378-6382 (1990); Devlin et al., Science, 249:404-406 (1990); Scott et al., Science, 249:386-388 (1990); and Ladner et al., U.S. Pat. No. 5,571,698). A basic concept of phage display methods is the establishment of a physical association between a polypeptide encoded by the phage DNA and a target antigen. This physical association is provided by the phage particle, which displays a polypeptide as part of a capsid enclosing the phage genome which encodes the polypeptide. The establishment of a physical association between polypeptides and their genetic material allows simultaneous mass screening of very large numbers of phage bearing different polypeptides. Phage displaying a polypeptide with affinity to a target antigen bind to the target antigen and these phage are enriched by affinity screening to the target antigen. The identity of polypeptides displayed from these phage can be determined from their respective genomes. Using these methods, a polypeptide identified as having a binding affinity for a desired target antigen can then be synthesized in bulk by conventional means (see, e.g., U.S. Pat. No. 6,057,098).

The antibodies that are generated by these methods can then be selected by first screening for affinity and specificity with the purified polypeptide antigen of interest and, if required, comparing the results to the affinity and specificity of the antibodies with other polypeptide antigens that are desired to be excluded from binding. The screening procedure can involve immobilization of the purified polypeptide antigens in separate wells of microtiter plates. The solution containing a potential antibody or group of antibodies is then placed into the respective microtiter wells and incubated for about 30 minutes to 2 hours. The microtiter wells are then washed and a labeled secondary antibody (e.g., an anti-mouse antibody conjugated to alkaline phosphatase if the raised antibodies are mouse antibodies) is added to the wells and incubated for about 30 minutes and then washed. Substrate is added to the wells and a color reaction will appear where antibody to the immobilized polypeptide antigen is present.

The antibodies so identified can then be further analyzed for affinity and specificity. In the development of immunoassays for a target protein (glycated hemoglobin), the purified target protein acts as a standard with which to judge the sensitivity and specificity of the immunoassay using the antibodies that have been selected. Because the binding affinity of various antibodies may differ, e.g., certain antibody combinations may interfere with one another sterically, assay performance of an antibody may be a more important measure than absolute affinity and specificity of that antibody.

Those skilled in the art will recognize that many approaches can be taken in producing antibodies or binding fragments and screening and selecting for affinity and specificity for the various polypeptides of interest, but these approaches do not change the scope of the present invention.

A. Polyclonal Antibodies

Polyclonal antibodies are preferably raised in animals by multiple subcutaneous (sc) or intraperitoneal (ip) injections of a polypeptide of interest and an adjuvant. It may be useful to conjugate the polypeptide of interest to a protein carrier that is immunogenic in the species to be immunized, such as, e.g., keyhole limpet hemocyanin, serum albumin, bovine thyroglobulin, or soybean trypsin inhibitor using a bifunctional or derivatizing agent. Non-limiting examples of bifunctional or derivatizing agents include maleimidobenzoyl sulfosuccinimide ester (conjugation through cysteine residues), N-hydroxysuccinimide (conjugation through lysine residues), glutaraldehyde, succinic anhydride, SOCl₂, and R₁N═C═NR, wherein R and R₁ are different alkyl groups.

Animals are immunized against the polypeptide of interest or an immunogenic conjugate or derivative thereof by combining, e.g., 100 μg (for rabbits) or 5 μg (for mice) of the antigen or conjugate with 3 volumes of Freund's complete adjuvant and injecting the solution intradermally at multiple sites. One month later, the animals are boosted with about 1/5 to 1/10 the original amount of polypeptide or conjugate in Freund's incomplete adjuvant by subcutaneous injection at multiple sites. Seven to fourteen days later, the animals are bled and the serum is assayed for antibody titer. Animals are typically boosted until the titer plateaus. Preferably, the animal is boosted with the conjugate of the same polypeptide, but conjugation to a different immunogenic protein and/or through a different cross-linking reagent may be used. Conjugates can also be made in recombinant cell culture as fusion proteins. In certain instances, aggregating agents such as alum can be used to enhance the immune response.

B. Monoclonal Antibodies

Monoclonal antibodies are generally obtained from a population of substantially homogeneous antibodies, i.e., the individual antibodies comprising the population are identical except for possible naturally-occurring mutations that may be present in minor amounts. Thus, the modifier “monoclonal” indicates the character of the antibody as not being a mixture of discrete antibodies. For example, monoclonal antibodies can be made using the hybridoma method described by Kohler et al., Nature, 256:495 (1975) or by any recombinant DNA method known in the art (see, e.g., U.S. Pat. No. 4,816,567).

In the hybridoma method, a mouse or other appropriate host animal (e.g., hamster) is immunized as described above to elicit lymphocytes that produce or are capable of producing antibodies which specifically bind to the polypeptide of interest used for immunization. Alternatively, lymphocytes are immunized in vitro. The immunized lymphocytes are then fused with myeloma cells using a suitable fusing agent, such as polyethylene glycol, to form hybridoma cells (see, e.g., Goding, Monoclonal Antibodies: Principles and Practice, Academic Press, pp. 59-103 (1986)). The hybridoma cells thus prepared are seeded and grown in a suitable culture medium that preferably contains one or more substances which inhibit the growth or survival of the unfused, parental myeloma cells. For example, if the parental myeloma cells lack the enzyme hypoxanthine guanine phosphoribosyl transferase (HGPRT), the culture medium for the hybridoma cells will typically include hypoxanthine, aminopterin, and thymidine (HAT medium), which prevent the growth of HGPRT-deficient cells.

Preferred myeloma cells are those that fuse efficiently, support stable high-level production of antibody by the selected antibody-producing cells, and/or are sensitive to a medium such as HAT medium. Examples of such preferred myeloma cell lines for the production of human monoclonal antibodies include, but are not limited to, murine myeloma lines such as those derived from MOPC-21 and MPC-11 mouse tumors (available from the Salk Institute Cell Distribution Center; San Diego, Calif.), SP-2 or X63-Ag8-653 cells (available from the American Type Culture Collection; Rockville, Md.), and human myeloma or mouse-human heteromyeloma cell lines (see, e.g., Kozbor, J. Immunol., 133:3001 (1984); and Brodeur et al., Monoclonal Antibody Production Techniques and Applications, Marcel Dekker, Inc., New York, pp. 51-63 (1987)).

The culture medium in which hybridoma cells are growing can be assayed for the production of monoclonal antibodies directed against the polypeptide of interest. Preferably, the binding specificity of monoclonal antibodies produced by hybridoma cells is determined by immunoprecipitation or by an in vitro binding assay, such as a radioimmunoassay (RIA) or an enzyme-linked immunoabsorbent assay (ELISA). The binding affinity of monoclonal antibodies can be determined using, e.g., the Scatchard analysis of Munson et al., Anal. Biochem., 107:220 (1980).

After hybridoma cells are identified that produce antibodies of the desired specificity, affinity, and/or activity, the clones may be subcloned by limiting dilution procedures and grown by standard methods (see, e.g., Goding, Monoclonal Antibodies: Principles and Practice, Academic Press, pp. 59-103 (1986)). Suitable culture media for this purpose include, for example, D-MEM or RPMI-1640 medium. In addition, the hybridoma cells may be grown in vivo as ascites tumors in an animal. The monoclonal antibodies secreted by the subclones can be separated from the culture medium, ascites fluid, or serum by conventional antibody purification procedures such as, for example, protein A-Sepharose, hydroxylapatite chromatography, gel electrophoresis, dialysis, or affinity chromatography.

DNA encoding the monoclonal antibodies can be readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as E. coli cells, simian COS cells, Chinese Hamster Ovary (CHO) cells, or myeloma cells that do not otherwise produce antibody, to induce the synthesis of monoclonal antibodies in the recombinant host cells. See, e.g., Skerra et al., Curr. Opin. Immunol., 5:256-262 (1993); and Pluckthun, Immunol Rev., 130:151-188 (1992). The DNA can also be modified, for example, by substituting the coding sequence for human heavy chain and light chain constant domains in place of the homologous murine sequences (see, e.g., U.S. Pat. No. 4,816,567; and Morrison et al., Proc. Natl. Acad. Sci. USA, 81:6851 (1984)), or by covalently joining to the immunoglobulin coding sequence all or part of the coding sequence for a non-immunoglobulin polypeptide.

In a further embodiment, monoclonal antibodies or antibody fragments can be isolated from antibody phage libraries generated using the techniques described in, for example, McCafferty et al., Nature, 348:552-554 (1990); Clackson et al., Nature, 352:624-628 (1991); and Marks et al., J. Mol. Biol., 222:581-597 (1991). The production of high affinity (nM range) human monoclonal antibodies by chain shuffling is described in Marks et al., BioTechnology, 10:779-783 (1992). The use of combinatorial infection and in vivo recombination as a strategy for constructing very large phage libraries is described in Waterhouse et al., Nuc. Acids Res., 21:2265-2266 (1993). Thus, these techniques are viable alternatives to traditional monoclonal antibody hybridoma methods for the generation of monoclonal antibodies. Human Antibodies

As an alternative to humanization, human antibodies can be generated. In some embodiments, transgenic animals (e.g., mice) can be produced that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Natl. Acad. Sci. USA, 90:2551 (1993); Jakobovits et al., Nature, 362:255-258 (1993); Bruggermann et al., Year in Immun., 7:33 (1993); and U.S. Pat. Nos. 5,591,669, 5,589,369, and 5,545,807.

Alternatively, phage display technology (see, e.g., McCafferty et al., Nature, 348:552-553 (1990)) can be used to produce human antibodies and antibody fragments in vitro, using immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B cell. Phage display can be performed in a variety of formats as described in, e.g., Johnson et al., Curr. Opin. Struct. Biol., 3:564-571 (1993). Several sources of V-gene segments can be used for phage display. See, e.g., Clackson et al., Nature, 352:624-628 (1991). A repertoire of V genes from unimmunized human donors can be constructed and antibodies to a diverse array of antigens (including self-antigens) can be isolated essentially following the techniques described in Marks et al., J. Mol. Biol., 222:581-597 (1991); Griffith et al., EMBO J., 12:725-734 (1993); and U.S. Pat. Nos. 5,565,332 and 5,573,905.

In certain instances, human antibodies can be generated by in vitro activated B cells as described in, e.g., U.S. Pat. Nos. 5,567,610 and 5,229,275.

C. Antibody Fragments

Various techniques have been developed for the production of antibody fragments. Traditionally, these fragments were derived via proteolytic digestion of intact antibodies (see, e.g., Morimoto et al., J. Biochem. Biophys. Meth., 24:107-117 (1992); and Brennan et al., Science, 229:81 (1985)). However, these fragments can now be produced directly using recombinant host cells. For example, the antibody fragments can be isolated from the antibody phage libraries discussed above. Alternatively, Fab′-SH fragments can be directly recovered from E. coli cells and chemically coupled to form F(ab′)2 fragments (see, e.g., Carter et al., BioTechnology, 10:163-167 (1992)). According to another approach, F(ab′)₂ fragments can be isolated directly from recombinant host cell culture. Other techniques for the production of antibody fragments will be apparent to those skilled in the art. In other embodiments, the antibody of choice is a single chain Fv fragment (scFv). See, e.g., PCT Publication No. WO 93/16185; and U.S. Pat. Nos. 5,571,894 and 5,587,458. The antibody fragment may also be a linear antibody as described, e.g., in U.S. Pat. No. 5,641,870. Such linear antibody fragments may be monospecific or bispecific.

D. Bispecific Antibodies

Bispecific antibodies are antibodies that have binding specificities for at least two different epitopes. Exemplary bispecific antibodies may bind to two different epitopes of the same polypeptide of interest. Other bispecific antibodies may combine a binding site for the polypeptide of interest with binding site(s) for one or more additional antigens. Bispecific antibodies can be prepared as full-length antibodies or antibody fragments (e.g., F(ab′)2 bispecific antibodies).

Methods for making bispecific antibodies are known in the art. Traditional production of full-length bispecific antibodies is based on the co-expression of two immunoglobulin heavy chain-light chain pairs, where the two chains have different specificities (see, e.g., Millstein et al., Nature, 305:537-539 (1983)). Because of the random assortment of immunoglobulin heavy and light chains, these hybridomas (quadromas) produce a potential mixture of 10 different antibody molecules, of which only one has the correct bispecific structure. Purification of the correct molecule is usually performed by affinity chromatography. Similar procedures are disclosed in PCT Publication No. WO 93/08829 and Traunecker et al., EMBO J., 10:3655-3659 (1991).

According to a different approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2, and CH3 regions. It is preferred to have the first heavy chain constant region (CH1) containing the site necessary for light chain binding present in at least one of the fusions. DNA encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains into one expression vector when the expression of at least two polypeptide chains in equal ratios results in high yields or when the ratios are of no particular significance.

In a preferred embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. This asymmetric structure facilitates the separation of the desired bispecific compound from unwanted immunoglobulin chain combinations, as the presence of an immunoglobulin light chain in only one half of the bispecific molecule provides for a facile way of separation. See, e.g., PCT Publication No. WO 94/04690 and Suresh et al., Meth. Enzymol., 121:210 (1986).

According to another approach described in U.S. Pat. No. 5,731,168, the interface between a pair of antibody molecules can be engineered to maximize the percentage of heterodimers which are recovered from recombinant cell culture. The preferred interface comprises at least a part of the CH3 domain of an antibody constant domain. In this method, one or more small amino acid side-chains from the interface of the first antibody molecule are replaced with larger side chains (e.g., tyrosine or tryptophan). Compensatory “cavities” of identical or similar size to the large side-chain(s) are created on the interface of the second antibody molecule by replacing large amino acid side-chains with smaller ones (e.g., alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Heteroconjugate antibodies can be made using any convenient cross-linking method. Suitable cross-linking agents and techniques are well-known in the art, and are disclosed in, e.g., U.S. Pat. No. 4,676,980.

Suitable techniques for generating bispecific antibodies from antibody fragments are also known in the art. For example, bispecific antibodies can be prepared using chemical linkage. In certain instances, bispecific antibodies can be generated by a procedure in which intact antibodies are proteolytically cleaved to generate F(ab′)2 fragments (see, e.g., Brennan et al., Science, 229:81 (1985)). These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody.

In some embodiments, Fab′-SH fragments can be directly recovered from E. coli and chemically coupled to form bispecific antibodies. For example, a fully humanized bispecific antibody F(ab′)2 molecule can be produced by the methods described in Shalaby et al., J. Exp. Med., 175: 217-225 (1992). Each Fab′ fragment was separately secreted from E. coli and subjected to directed chemical coupling in vitro to form the bispecific antibody.

Various techniques for making and isolating bispecific antibody fragments directly from recombinant cell culture have also been described. For example, bispecific antibodies have been produced using leucine zippers. See, e.g., Kostelny et al., J. Immunol., 148:1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers.

The “diabody” technology described by Hollinger et al., Proc. Natl. Acad. Sci. USA, 90:6444-6448 (1993) has provided an alternative mechanism for making bispecific antibody fragments. The fragments comprise a heavy chain variable domain (VH) connected to a light chain variable domain (VL) by a linker which is too short to allow pairing between the two domains on the same chain. Accordingly, the VH and VL domains of one fragment are forced to pair with the complementary VL and VH domains of another fragment, thereby forming two antigen binding sites. Another strategy for making bispecific antibody fragments by the use of single-chain Fv (sFv) dimers is described in Gruber et al., J. Immunol., 152:5368 (1994).

Antibodies with more than two valencies are also contemplated. For example, trispecific antibodies can be prepared. See, e.g., Tutt et al., J. Immunol., 147:60 (1991).

E. Antibody Purification

When using recombinant techniques, antibodies can be produced inside an isolated host cell, in the periplasmic space of a host cell, or directly secreted from a host cell into the medium. If the antibody is produced intracellularly, the particulate debris is first removed, for example, by centrifugation or ultrafiltration. Carter et al., BioTech., 10:163-167 (1992) describes a procedure for isolating antibodies which are secreted into the periplasmic space of E. coli. Briefly, cell paste is thawed in the presence of sodium acetate (pH 3.5), EDTA, and phenylmethylsulfonylfluoride (PMSF) for about 30 min. Cell debris can be removed by centrifugation. Where the antibody is secreted into the medium, supernatants from such expression systems are generally concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The antibody composition prepared from cells can be purified using, for example, hydroxylapatite chromatography, gel electrophoresis, dialysis, and affinity chromatography. The suitability of protein A as an affinity ligand depends on the species and isotype of any immunoglobulin Fc domain that is present in the antibody. Protein A can be used to purify antibodies that are based on human γ1, γ2, or γ4 heavy chains (see, e.g., Lindmark et al., J. Immunol. Meth., 62:1-13 (1983)). Protein G is recommended for all mouse isotypes and for human γ3 (see, e.g., Guss et al., EMBO J., 5:1567-1575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a CH3 domain, the Bakerbond ABX™ resin (J. T. Baker; Phillipsburg, N.J.) is useful for purification. Other techniques for protein purification such as fractionation on an ion-exchange column, ethanol precipitation, reverse phase HPLC, chromatography on silica, chromatography on heparin SEPHAROSE™, chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody of interest and contaminants may be subjected to low pH hydrophobic interaction chromatography using an elution buffer at a pH between about 2.5-4.5, preferably performed at low salt concentrations (e.g., from about 0-0.25 M salt).

### III. Specific Antibodies

In one aspect, an antibody specific for the glycated form of HbA1c is used. This antibody does not cross-react with HbA₀. In one aspect, this antibody is commercially available from Mybiosource.com having Catalog # MBS31270 and is a monoclonal IgG1 that reacts with Hemoglobin A1c (HbA1c) with no cross-reactivity. In another aspect, a commercially available antibody from GeneTex Cat No. GTX42177, which is a Hemoglobin A1c (HbA1c) antibody with no cross reactivity. In one aspect, an acceptor fluorophore can be conjugated to these antibodies.

In one aspect, a mouse monoclonal antibody from Lifespan Biosciences, which is a hemoglobin antibody (clone HB11-201.11) IHC-Plus™ LS-B4914 or (clone M1709Hg2) IHC-Plus™ LS-B11162 or LS-C194323 having human reactivity is used.

### IV. Device

Various instruments and devices are suitable for use in the present disclosure. Many spectrophotometers have the capability to measure fluorescence. Fluorescence is the molecular absorption of light energy at one wavelength and its nearly instantaneous re-emission at another, longer wavelength. Some molecules fluoresce naturally, and others must be modified to fluoresce.

A fluorescence spectrophotometer or fluorometer, fluorospectrometer, or fluorescence spectrometer measures the fluorescent light emitted from a sample at different wavelengths, after illumination with light source such as a xenon flash lamp. Fluorometers can have different channels for measuring differently-colored fluorescent signals (that differ in their wavelengths), such as green and blue, or ultraviolet and blue, channels. In one aspect, a suitable device includes an ability to perform a time-resolved fluorescence resonance energy transfer (FRET) experiment.

Suitable fluorometers can hold samples in different ways, including cuvettes, capillaries, Petri dishes, and microplates. The assays described herein can be performed on any of these types of instruments. In certain aspects, the device has an optional microplate reader, allowing emission scans in up to 384-well plates, Others models suitable for use hold the sample in place using surface tension.

Time-resolved fluorescence (TRF) measurement is similar to fluorescence intensity measurement. One difference, however, is the timing of the excitation/measurement process. When measuring fluorescence intensity, the excitation and emission processes are simultaneous: the light emitted by the sample is measured while excitation is taking place. Even though emission systems are very efficient at removing excitation light before it reaches the detector, the amount of excitation light compared to emission light is such that fluorescent intensity measurements exhibit elevated background signals. The present disclosure offers a solution to this issue. Time resolve FRET relies on the use of specific fluorescent molecules that have the property of emitting over long periods of time (measured in milliseconds) after excitation, when most standard fluorescent dyes (e.g. fluorescein) emit within a few nanoseconds of being excited. As a result, it is possible to excite cryptate lanthanides using a pulsed light source (e.g., Xenon flash lamp or pulsed laser), and measure after the excitation pulse.

As the donor and acceptor fluorescent compounds attached to antibody 1 and 2 move closer together, an energy transfer is caused from the donor compound to the acceptor compound, resulting in a decrease in the fluorescence signal emitted by the donor compound and an increase in the signal emitted by the acceptor compound, and vice-versa. The majority of biological phenomena involving interactions between different partners will therefore be able to be studied by measuring the change in FRET between 2 fluorescent compounds coupled with compounds which will be at a greater or lesser distance, depending on the biological phenomenon in question.

The FRET signal can be measured in different ways: measurement of the fluorescence emitted by the donor alone, by the acceptor alone or by the donor and the acceptor, or measurement of the variation in the polarization of the light emitted in the medium by the acceptor as a result of FRET. One can also include measurement of FRET by observing the variation in the lifetime of the donor, which is facilitated by using a donor with a long fluorescence lifetime, such as rare earth complexes (especially on simple equipment like plate readers). Furthermore, the FRET signal can be measured at a precise instant or at regular intervals, making it possible to study its change over time and thereby to investigate the kinetics of the biological process studied.

In certain aspects, the device disclosed in PCT/IB2019/051213, filed Feb. 14, 2019 is used, which is hereby incorporated by reference. That disclosure in that application generally relates to analyzers that can be used in point-of-care (POC) settings to measure the absorbance and fluorescence of a sample with minimal or no user handling or interaction. The disclosed analyzers provide advantageous features of more rapid and reliable analyses of samples having properties that can be detected with each of these two approaches. For example, it can be beneficial to quantify both the fluorescence and absorbance of a blood sample being subjected to a diagnostic assay. In some analytical workflows, the hematocrit of a blood sample can be quantified with an absorbance assay, while the signal intensities measured in a FRET assay can provide information regarding other components of the blood sample.

One apparatus disclosed in PCT/IB2019/051213 is useful for detecting an emission light from a sample, and absorbance of a transillumination light by the sample, which comprises a first light source configured to emit an excitation light having an excitation wavelength. The apparatus further comprises a second light source configured to transilluminate the sample with the transillumination light. The apparatus further comprises a first light detector configured to detect the excitation light, and a second light detector configured to detect the emission light and the transillumination light. The apparatus further comprises a dichroic mirror configured to (1) epi-illuminate the sample by reflecting at least a portion of the excitation light, (2) transmit at least a portion of the excitation light to the first light detector, (3) transmit at least a portion of the emission light to the second light detector, and (4) transmit at least a portion of the transillumination light to the second light detector.

One suitable cuvette for use in the present disclosure is disclosed in PCT/IB2019/051215, filed Feb. 14, 2019. One of the provided cuvettes comprises a hollow body enclosing an inner chamber having an open chamber top. The cuvette further comprises a lower lid having an inner wall, an outer wall, an open lid top, and an open lid bottom. At least a portion of the lower lid is configured to fit inside the inner chamber proximate to the open chamber top. The lower lid comprises one or more (e.g., two or more) containers connected to the inner wall, wherein each of the containers has an open container top. In certain aspects, the lower lid comprises two or more such containers. The lower lid further comprises a securing means connected to the hollow body. The cuvette further comprises an upper lid wherein at least a portion of the upper lid is configured to fit inside the lower lid proximate to the open lid top.

### V. Examples

### Example 1

This example shows a solution phase homogenous time resolved FRET assay to detect HbA1c levels in blood.

Levels of glycated Hemoglobin (HbA1C) can be used as an aid in diagnosing diabetes and also measuring effect of diabetes treatments. Fluorescence resonance energy transfer (FRET) is a process in which a donor molecule in excited state transfers its excitation energy through dipole-dipole coupling to an acceptor fluorophore, when the two are brought into close proximity (typically less than 10 nm). Upon excitation at a characteristic wavelength, the energy absorbed by the donor is transferred to the acceptor, which in turn emits the energy. The level of light emitted from the acceptor fluorophore is proportional to the degree of donor acceptor complex formation.

Biological sample materials are prone to auto-fluorescence, which can be minimized by utilizing time-resolved fluorometry (TRF). TRF takes advantage of unique rare earth elements called lanthanides, such as europium and terbium, which have exceptionally long fluorescence emission half-lives. Time-resolved FRET (TR-FRET) unites the properties of TRF and FRET, which is especially advantageous when analyzing biological samples.

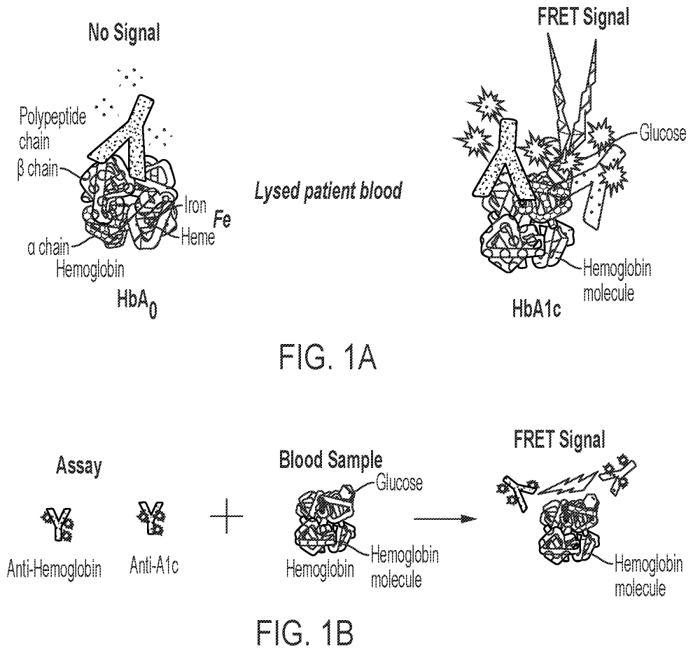

In one aspect, the anti-HbA₀ antibody is labeled with a donor fluorophore and a second anti-HbA1c antibody is labeled with an acceptor fluorophore, thus TR-FRET occurs only in the presence of glycated hemoglobin (FIG. 1A-B). The increase in FRET signal of the acceptor is proportional to the percentage of glycated hemoglobin present in the patient's blood as interpolated from a known amount of HbA1c glycation (FIG. 2A-B). The H22TRENIAM-5LIO-NHS is used to label the HbA₀ antibody (FIG. 3). Lumi4 has 4 spectrally distinct peaks, at about 490 nm, about 545 nm, about 580 nm, and about 620 nm, which can be used for energy transfer (FIG. 4). The acceptor molecules that can be used include, but are not limited to, AlexaFluor 488, AlexaFluor 546 and AlexaFluor 647 (FIG. 5). Donor and acceptor fluorophores are conjugated using primary amines on antibodies. Donor and acceptor fluorophores are conjugated using primary amines on anti-Hb antibodies.

### Example 2

This example illustrates a calculation of total hemoglobin concentration (ctHb) from hematocrit levels (Hct).

The total Hb concentration can be measured by measuring the amount of hematocrit (Hct). Hematocrit is the ratio of the volume of packed red blood cells to the total blood volume. It is also known as the packed cell volume, or PCV. There is a linear relationship between hematocrit and the concentration of hemoglobin (ctHb). The relationship can be expressed as follows:

Hct (%)=(0.0485×ctHb (mmol/L)+0.0083)×100

(Kokholm G. Simultaneous measurements of blood pH, pCO2, pO2 and concentrations of hemoglobin and its derivatives—a multicenter study. Radiometer publication AS107. Copenhagen: Radiometer Medical A/S, 1991).

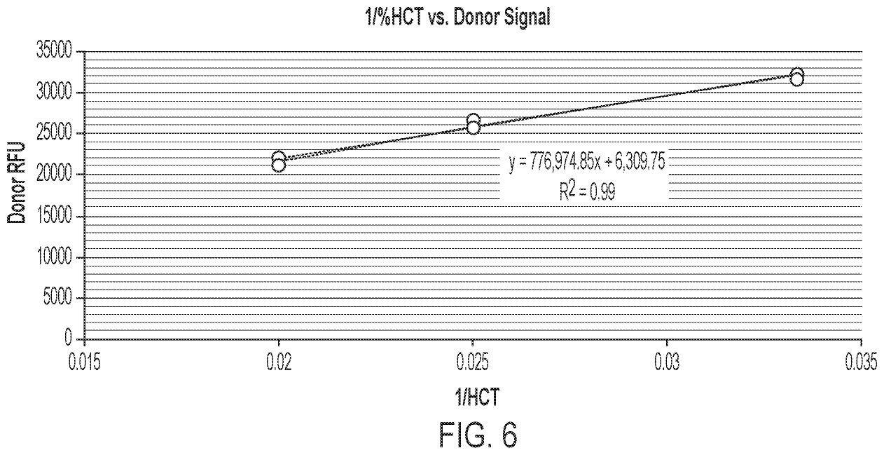

FIG. 6 illustrates a standard curve of Hct (%) samples showing the effect of fluorescence on a donor signal. Using the equation above, it is possible to calculate the total amount of Hb (ctHb).

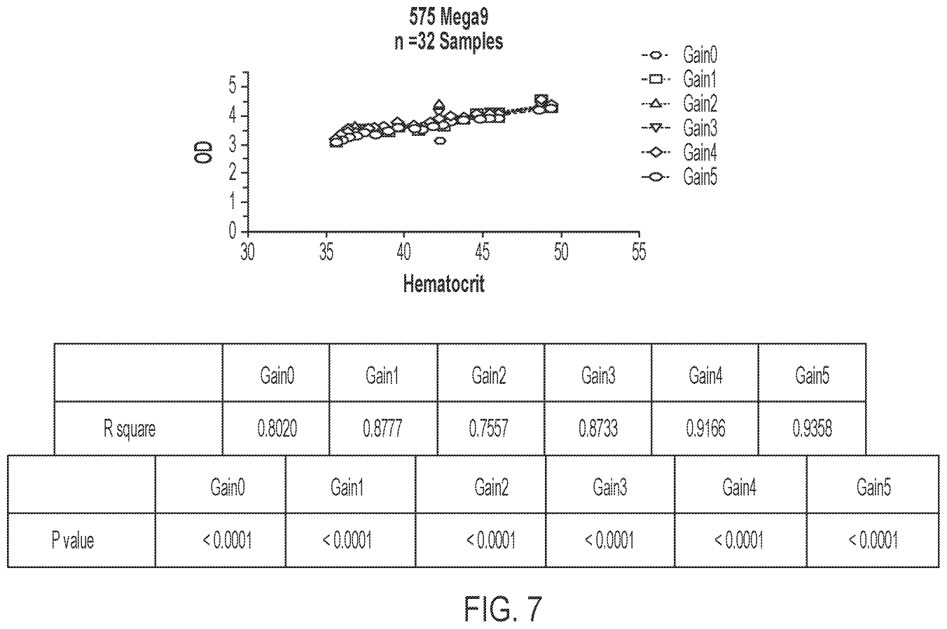

FIG. 7 illustrates that the hematocrit level can be determined using absorption of a known amount of donor fluorophore.

### Example 3

This example illustrates a calculation of total hemoglobin concentration using a CO-oximetery method.

The measurement of ctHb by CO-oximetry is based on the fact that hemoglobin and all its derivatives are colored proteins which absorb light at specific wavelengths and thus have a characteristic absorbance spectrum. Beer-Lambert's law dictates that absorbance of a single compound is proportional to the concentration of that compound.

In a CO-oximeter absorbance measurement of a hemolyzed blood sample, light is irradiated at multiple wavelengths across a range that hemoglobin species absorb light (520-620 nm) and software calculates the concentration of each of the hemoglobin derivatives (HHb, O₂Hb, MetHb and COHb). Total hemoglobin (ctHb) is the calculated sum of these derivatives.

### Example 4

This example illustrates a head to head comparison between the currently disclosed methods and a point-of-care (POC) device by Afinion.

Point-of-care (POC) testing is becoming increasingly valuable in health care delivery, and it is important that the devices used meet the same quality criteria as main laboratory analyzers. POC HbA1c measurements can expedite diagnostic decisions and medical interventions provided they meet performance standards.

Analytical performance of the disclosed method (Prosice, inventive) was compared to Afinion POC analyzer. 14 known samples with known HbA1c values between 4.9% and 9.9% were used. The samples were measured in the Afinion analyzer and then compared to the measurements using the inventive method. The measurements using the inventive methods were performed in duplicate. Whole blood was used at a final concentration of 0.25%. The anti-A1c antibody is conjugated to a cryptate and the anti-Hb antibody is conjugated to Alexa 647.

The results are tabulated in the Table 1 below.

TABLE 1

| | % A1c | Average % | Std | % | % |
|---|---|---|---|---|---|
| Sample | Afinion | A1c Procise (N = 2) | Dev | CV | Accuracy |
| SDBB-4.9 | 4.9 | 4.8 | 0.05 | 0.9% | 98.6% |
| SDBB-5.1 | 5.1 | 5.2 | 0.09 | 1.7% | 102.8% |
| SDBB-5.2 | 5.2 | 5.32 | 0.03 | 0.5% | 102.3% |
| SDBB-5.3 | 5.3 | 5.4 | 0.16 | 2.9% | 101.8% |
| SDBB-5.4 | 5.4 | 5.32 | 0.03 | 0.5% | 98.5% |
| SDBB-5.7 | 5.7 | 5.6 | 0.07 | 1.2% | 97.7% |
| SDBB-6.3 | 6.3 | 6.1 | 0.14 | 2.3% | 97.1% |
| SDBB-6.7 | 6.7 | 6.6 | 0.13 | 1.9% | 98.6% |
| SDBB-7.7 | 7.7 | 7.8 | 0.01 | 0.1% | 100.7% |
| SDBB-9.9 | 9.9 | 10.0 | 0.10 | 1.0% | 101.4% |
| SDBB-5.5 | 5.5 | 5.5 | 0.17 | 3.0% | 100.4% |
| SDBB-5.8 | 5.8 | 5.7 | 0.10 | 1.8% | 98.7% |
| SDBB-6.0 | 6.0 | 5.7 | 0.09 | 1.5% | 95.0% |
| SDBB-6.1 | 6.1 | 6.0 | 0.11 | 1.8% | 98.4% |

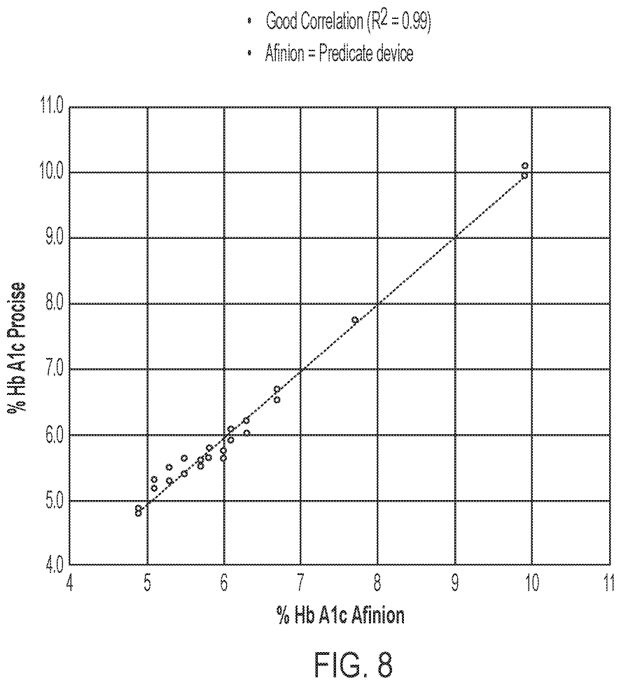

FIG. 8 shows the comparison of HbA1c values obtained with the present methods and the Afinion measured values. FIG. 8 also shows the linear regression line. In general, the R² coefficient of determination is a statistical measure of how well the regression predictions approximate the real data points. A R² of 1 indicates that the regression predictions perfectly fit the data. Here, the R² is equal to 0.99 showing excellent correlation of the inventive methods.

### Example 5

This example illustrates a head to head comparison between the currently disclosed methods (inventive) and the Bio-Rad D-100 system (comparator). The BioRad D-100 is based on the separation of Hb fractions by ion-exchange HPLC. The samples were measured in the Bio-Rad D-100 system and then compared to the measurements using the inventive method. The measurements using the inventive methods were performed in duplicate. Whole blood was used at a final concentration of 0.25%. The anti-A1c antibody is conjugated to a cryptate and the anti-Hb antibody is conjugated to Alexa 647. The results are tabulated in Table 2 below.

TABLE 2

| | | | Average | | | |
|---|---|---|---|---|---|---|
| | | % A1c | % A1c | | | |
| | | BioRad | Procise | Std | % | % |
| Sample | Hb Type | D-100 | (N = 2) | Dev | CV | Accuracy |
| Biorad-8.3 | Nonvariant | 8.3 | 8.2 | 0.01 | 0.1% | 99.1% |
| Biorad-6.4 | Nonvariant | 6.4 | 6.5 | 0.04 | 0.6% | 100.8% |
| Biorad-9.1 | Nonvariant | 9.1 | 9.2 | 0.03 | 0.3% | 102.1% |
| Biorad-5.6 | AC | 5.6 | 5.4 | 0.05 | 0.9% | 96.1% |
| Biorad-5.2 | AD | 5.2 | 6.3 | 0.12 | 1.9% | 104.8% |
| Biorad-5.2 | AE | 5.2 | 5.8 | 0.16 | 2.7% | 102.2% |
| Biorad-5.2 | AS | 5.2 | 5.4 | 0.04 | 0.7% | 100.2% |
| Biorad-8.0 | AF | 8.0 | 8.0 | 0.06 | 0.8% | 100.2% |

Table 2 shows the comparison of HbA1c values obtained with the present methods and BioRad D-100 measured values. In most instances, the coefficient of variation (% CV), also known as relative standard deviation, (standard deviation [SD]/[mean]×100), is less than 1 indicating low-variance.

### Example 6

This example illustrates head to head comparisons between the currently disclosed methods and commercial calibration standards including (i) Pointe Scientific Standards; (ii) BioRad/Lyphochek standards; and measured samples from blood banked samples including a comparison of (iii) Afinion measured samples and (iv) D-100 (BioRad) measured samples.

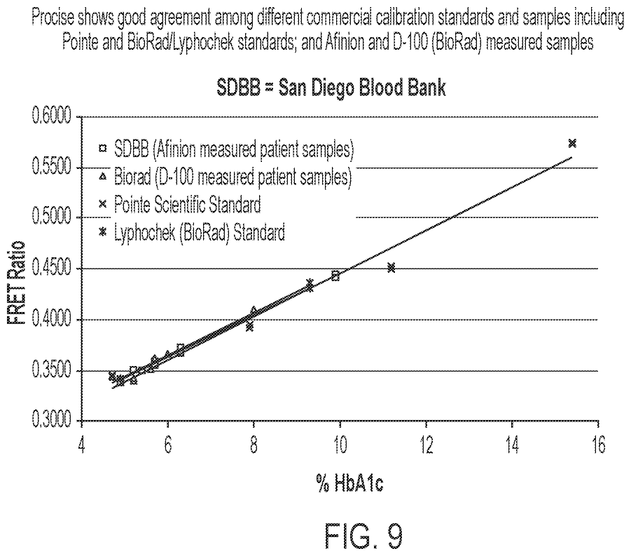

FIG. 9 shows good agreement of the disclosed methods measuring HbA1c when compared to HbA1c values using commercial calibration standards and measured samples.

Although the foregoing disclosure has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference.

1. A method for measuring the amount of glycated hemoglobin (HbA1c) in a sample, the method comprising:
contacting the sample with an anti-hemoglobin (HbA₀) antibody labeled with a first fluorophore, wherein the anti-hemoglobin (HbA₀) antibody also binds glycated hemoglobin (HbA1c);
contacting the sample with an anti-glycated hemoglobin (HbA1c) antibody labeled with a second fluorophore;
incubating the sample for a time sufficient to obtain a dual labeled glycated hemoglobin (HbA1c); and
exciting the sample have dual labeled glycated hemoglobin (HbA1c) using a light source to detect fluorescence emission signal associated with fluorescence resonance energy transfer (FRET).

2. The method according to claim 1, wherein total hemoglobin is measured in the sample.

3. The method according to claim 1, wherein the sample includes red blood cells.

4. The method according to claim 1, wherein the red blood cells are from whole blood.

5. The method according to claim 1, wherein the red blood cells are lysed.

6. The method according to claim 1, wherein the sample does not include red blood cells.

7. The method according to claim 1, wherein the FRET emission signal is a time resolved FRET emission signal.

8. The method according to claim 1, wherein the first fluorophore is a FRET energy donor.

9. The method according to claim 8, wherein the FRET energy donor is a terbium cryptate.

10. The method according to claim 1, wherein the second fluorophore is a FRET acceptor.

11. The method according to claim 10, wherein the acceptor is a member selected from the group consisting of fluorescein-like (green zone), Cy5, DY-647, Alexa Fluor 488, Alexa Fluor 546, Allophycocyanin (APC), Phycoeruythrin (PE) and Alexa Fluor 647.

12. The method according to claim 1, wherein the acceptor compound is Alexa Fluor 647.

13. The method according to claim 1, wherein the excitation wavelength is between about 300 nm to about 400 nm.

14. The method according to claim 1, wherein the emission wavelength is about 450 nm to 700 nm.

15. The method according to claim 1, wherein the glycated hemoglobin (HbA1c) value is less than 5.7%.

16. The method according to claim 1, wherein the glycated hemoglobin (HbA1c) value is between than 5.7 to 6.4%.

17. The method according to claim 1, wherein the glycated hemoglobin (HbA1c) value is at least 6.4%.

18. A method for measuring the amount of glycated hemoglobin (HbA1c) in vitro in a sample, the method comprising:
obtaining a sample from a subject;
contacting the sample with an anti-hemoglobin (HbA₀) antibody labeled with a first fluorophore, wherein the anti-hemoglobin (HbA₀) antibody also binds glycated hemoglobin (HbA1c);
contacting the sample with an anti-glycated hemoglobin (HbA1c) antibody labeled with a second fluorophore;
incubating the sample for a time sufficient to obtain a dual labeled glycated hemoglobin (HbA1c); and
exciting the sample have dual labeled glycated hemoglobin (HbA1c) using a light source to detect fluorescence emission signal associated with fluorescence resonance energy transfer (FRET), to determine the amount of glycated hemoglobin (HbA1c) in the sample.

19. The method according to claim 18, wherein the FRET energy donor is a terbium cryptate.

20. The method according to claim 18, wherein the second fluorophore is a FRET acceptor.
